(19) United States
(12) Patent Application Publication
(10) Pub. No.: US 2017/0042713 A1
(43) Pub. Date: Feb. 16, 2017

(54) SYSTEM AND METHODS FOR MOBILE MEDICAL MONITORING

(71) Applicants: Arto V. NURMIKKO, Providence, RI (US); David Christopher HEELAN, Providence, RI (US); David Allenson BORTON, Providence, RI (US); Jacob KOMAR, Providence, RI (US); David ROSLER, Providence, RI (US); John D. SIMERAL, East Greenwich, RI (US); Brown University, Providence, RI (US)

(72) Inventors: Arto V. NURMIKKO, Providence, RI (US); Christopher David HEELAN, Providence, RI (US); David Allenson BORTON, Providence, RI (US); Jacob KOMAR, Providence, RI (US); David ROSLER, Providence, RI (US); John D. SIMERAL, East Greenwich, RI (US)

(21) Appl. No.: 15/304,382

(22) PCT Filed: Apr. 13, 2015
(86) PCT No.: PCT/US15/25595

§ 371 Date: Oct. 14, 2016

Related U.S. Application Data
(60) Provisional application No. 61/979,127, filed on Apr. 14, 2014.

(51) Int. Cl. A61F 4/00; A61B 5/0205; A61B 5/03; A61B 5/04; A61B 5/11; A61B 5/145; A61B 5/00; A61N 1/36; A61B 5/0482; A61F 2/14; A61M 5/172; A61F 2/04; A61N 1/39; A61F 2/26; A61F 2/44; G06F 3/01; A61B 5/0404; G06F 19/00; A61B 5/16

(52) U.S. Cl. CPC A61F 4/00; A61B 5/165; A61B 5/02055; A61B 5/031; A61B 5/04012; A61B 5/1113; A61B 5/145; A61B 5/4851; A61N 1/36; A61B 5/0006; A61B 5/0482; A61F 2/14; A61M 5/1723; A61F 2/04; A61N 1/39; A61F 2/26; A61F 2/44; G06F 3/015; A61B 5/0404; G06F 19/3418; A61B 5/02438

(57) ABSTRACT

A portable monitoring device for whole-body monitoring can include a receiver configured to receive wireless signals representing real-time neural activity from a neural sensor and to process the wireless signals into digital signals. The portable monitoring device can also include a first processor coupled to the receiver configured to receive the digital signals from the receiver and a programmable processor coupled to the first processor. The programmable processor can be configured to process the digital signals and generate a mapping of the neural activity into a person's behavior, a person's mood, a person's health condition, a person's memory, or a person's intentions.

### CROSS-REFERENCE TO RELATED APPLICATION

This application claims the benefit under 35 U.S.C. §119(e) of U.S. Provisional Patent Application No. 61/979,127, entitled “Systems and Methods for Mobile Medical Monitoring,” filed Apr. 14, 2014, the entire contents of which are incorporated by reference herein.

This application is also related to:

U.S. Provisional Application No. 61/810,950, entitled “OPTOELECTRONIC DEVICE TO WRITE-IN AND READ-OUT ACTIVITY IN BRAIN CIRCUITS,” filed on Apr. 11,2013; and

U.S. application Ser. No. 14/028,178, entitled “IMPLANTABLE WIRELESS NEURAL DEVICE,” filed on Sep. 16, 2013,

the contents of which are incorporated herein by reference in their entirety.

This invention was made with government support under R01 EB740101 awarded by National Institutes of Health. The government has certain rights in the invention.

### BACKGROUND

Field of the Invention

This invention relates to portable devices for implementing mobile monitoring systems and methods for using such portable devices.

Discussion of Related Art

Brain-machine interface systems allow people with severe disabilities (e.g. paralysis) to partially or fully restore lost functions that may have been the result, for example, of injuries or diseases. However, people with severe disabilities have been limited to using brain-machine interface systems within controlled and stationary environments or research settings. For example, people using brain machine interface systems usually lie in hospital beds or are restricted within the bounds of their home, where large and elaborate systems can implement the brain-machine interfaces. The computational complexity of translating neural signals into device control commands has prevented this technology from becoming mobile because of the complexity and time required to complete the brain-model based calculations.

However, it is highly desirable to provide people with additional mobility and still enable them to benefit from brain-machine interface systems in their everyday lives. Therefore, there is a need for implementing portable brain-machine interface systems that can handle the complex and heavy computation requirements of sensing and decoding neural signals, and translating the neural signals into actionable commands that can be executed by monitoring systems.

It is also desirable to combine information from sources other than the brain, for example, from heart rate sensors, temperature sensors, or respiratory effort sensors, to implement a whole-body monitoring system that can generate actionable commands. Prior art whole-body monitoring systems constrain the people within controlled environments and limit their mobility.

### SUMMARY

According to aspects of the disclosure, whole-body monitoring systems can receive and process information from different sources sensing vital signals from different body parts and the brain, and translate the signals into commands, without severely constraining the system user's mobility and activities. The disclosed systems and methods can be implemented on portable devices that can communicate with different interfaces and can process information from various sources.

The portable monitoring devices of the present invention can provide real-time control of mobile machines and other devices such as wheelchairs or functional electrical stimulators in a mobile setting. As a result, the disclosed systems and methods can enable people to use these technologies to aid in everyday activities. The portable device may be an external device with a removable power source or it may be implanted with a rechargeable battery that chat can be charged via wireless power transfer from outside the body.

According to aspects of the disclosure, a portable monitoring device for whole body monitoring can include a receiver configured to receive wireless signals representing real-time neural activity from a neural sensor configured to sample neural signals from a person and process the wireless signals into corresponding digital signals. The portable monitoring device can also include a first processor configured to receive the digital signals from the receiver, process the digital signals to generate processed digital signals, and generate a mapping of the neural activity into at least one of a person's behavior, a person's mood, a person's health condition, a person's memory, and a person's intentions. The portable monitoring device can also include a second processor coupled to the first processor configured to receive the digital signals and the processed digital signals data from the first processor, and prepare at least one of the digital signals and the processed digital signals data for transmission to at least one peripheral device coupled to the portable monitoring device.

According to aspects of the disclosure a method for whole body monitoring using a portable monitoring device can include the steps of receiving, by a receiver of the portable monitoring device, wireless signals representing real-time neural activity from a neural sensor configured to sample neural signals from a person and processing, by the receiver, the wireless signals into corresponding digital signals. The method can also include the steps of receiving, by a first processor of the portable monitoring device, the digital signals from the receiver, processing, by the first processor, the digital signals to generate processed digital signals; generating, by the first processor, a mapping of the neural activity into at least one of a person's behavior, a person's mood, a person's health condition, a person's memory, and a person's intentions. The method can also include the steps of receiving, by a second processor of the portable monitoring device coupled to the first processor, the digital signals and the processed digital signals data from the first processor and preparing, by the second processor, at least one of the digital signals and the processed digital signals data for transmission to at least one peripheral device coupled to the portable monitoring device. The method can also include the steps of receiving peripheral device signals from the at least one peripheral device, sending the received peripheral device signals to the first processor for processing, receiving, from the first processor, the processed peripheral device signals, and preparing the processed peripheral device signals for transmission to the at least one peripheral device.

### BRIEF DESCRIPTION OF THE SEVERAL VIEWS OF THE DRAWINGS

For a more complete understanding of various embodiments of the present invention, reference is now made to the following descriptions taken in connection with the accompanying drawings, in which:

FIG. 1 shows a prior art brain-machine interface system.

FIG. 2 shows an exemplary architecture of a prior art brain-machine interface system.

FIG. 3 shows an exemplary whole-body monitoring system according to aspects of the present disclosure.

FIG. 4 shows exemplary components of a whole-body monitoring system according to aspects of the present disclosure.

FIG. 5 shows an exemplary implementation of a whole-body monitoring system according to aspects of the present disclosure using wireless components.

FIG. 6 shows an exemplary implementation of a whole-body monitoring system according to aspects of the present disclosure using wired and wireless components.

FIG. 7 shows an exemplary architecture for a portable monitoring device according to aspects of the present disclosure.

FIG. 8 shows exemplary components of a whole-body monitoring system according to aspects of the present disclosure.

### DETAILED DESCRIPTION

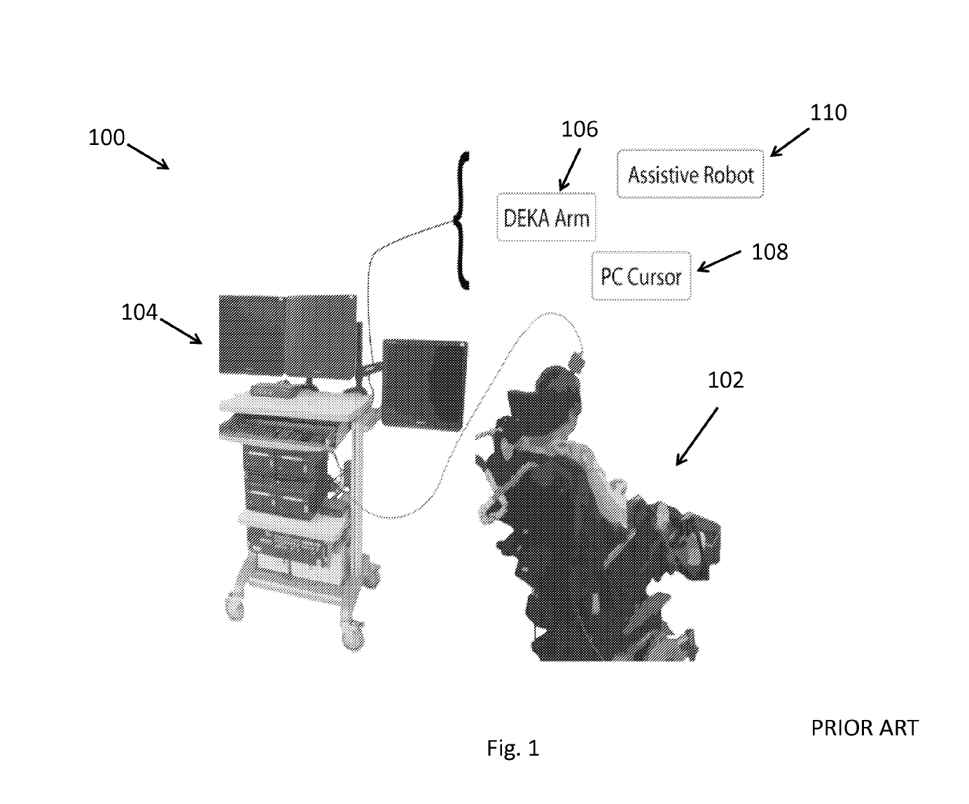

FIG. 1 shows a prior art brain-machine interface system 100. Specifically, FIG. 1 shows a person sitting in a wheelchair 102 and a processor system 104 for processing signals sensed from the person's brain. The processor system 104 can process the sensed signals from the person's head and can control different devices and interfaces, such as a prosthetic arm 106, a PC cursor 108, and an assistive robot 110. The prosthetic arm, PC cursor, and assistive robot represent examples of devices the user could control using the portable device by thinking about controlling them.

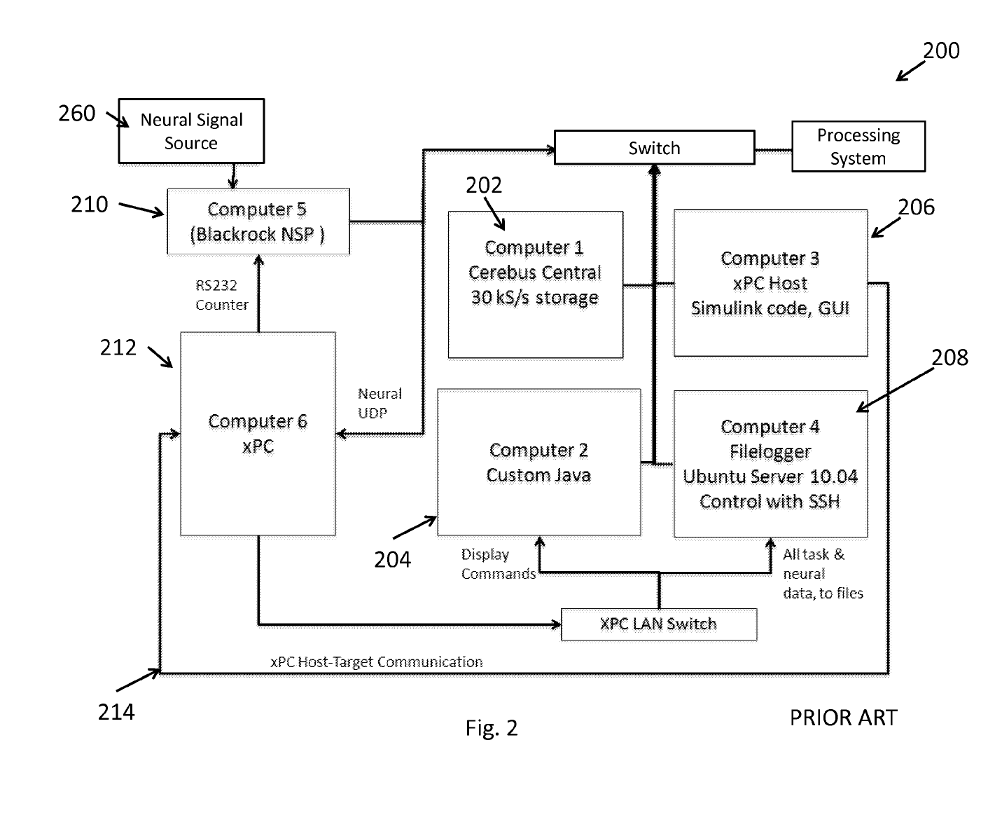

FIG. 2 shows an exemplary architecture 200 of the processor system of FIG. 1. The processing system is composed of six different personal computers (202, 204, 206, 208, 210, 212) communicating over a local network 214 that are responsible for performing the calculations necessary to translate sensor data (e.g. neural data 216) into meaningful control commands for downstream effectors. Additionally, the system handles the storage of the data as well as the training of the processing model.

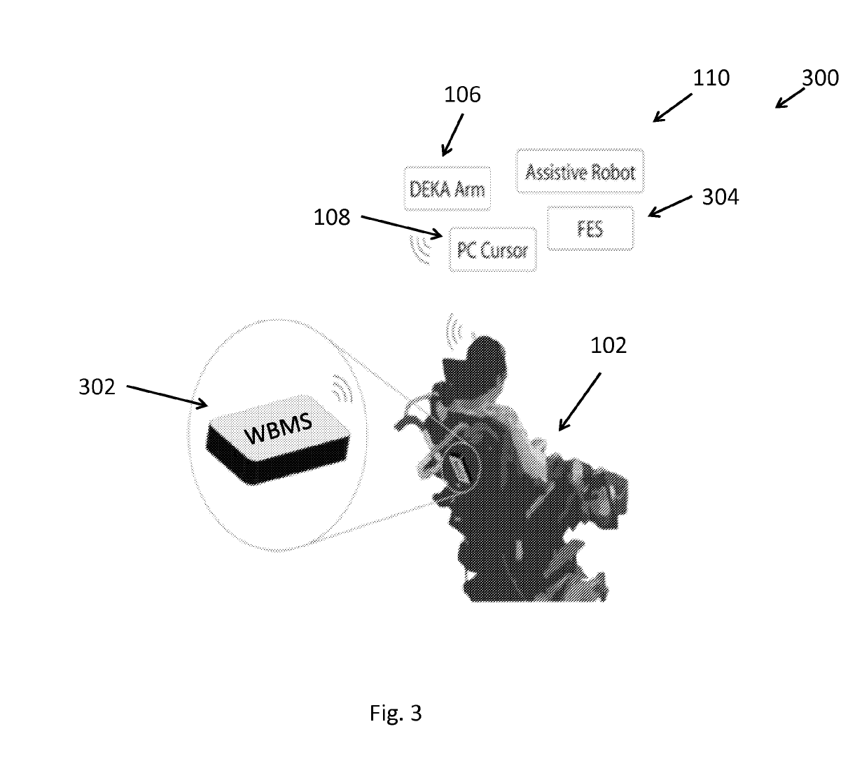

FIG. 3 shows an exemplary architecture of a whole-body monitoring system (WBMS) 300. The portable monitoring device 302 can be carried by a person 102, or can be attached, for example to a wheelchair or a battery-powered chair, as shown in FIG. 3. For example, the portable monitoring device can have a form factor similar to a smartphone. The portable monitoring device 302 can process the sensed signals from a person 102, for example, from neural signals from a person's head. A sensor on or embedded in a person's head can collect data in multiple channels at high sampling rates, for example, 100 channels at 24 KHz, and transmit it via wireless means to the portable monitoring device 302. The portable monitoring device 302 can control different devices and interfaces, such as a prosthetic arm 106, a PC cursor 108, an assistive robot 110, and an “FES” block 304. The “FES” block 304 represents a functional electrical stimulator device, which can electrically stimulate the user's muscles to contract thereby enabling motion of paralyzed limbs. For example, the FES block 304 can allow a paralyzed user to regain movement of their own limbs by thinking. According to aspects of the disclosure, the portable monitoring device 302 can control the devices and interfaces by transmitting commands as wireless signals, which can be received by receivers in the devices and interfaces. The portable monitoring device 302 can be wall- and/or battery-operated, with a replaceable and/or rechargeable battery.

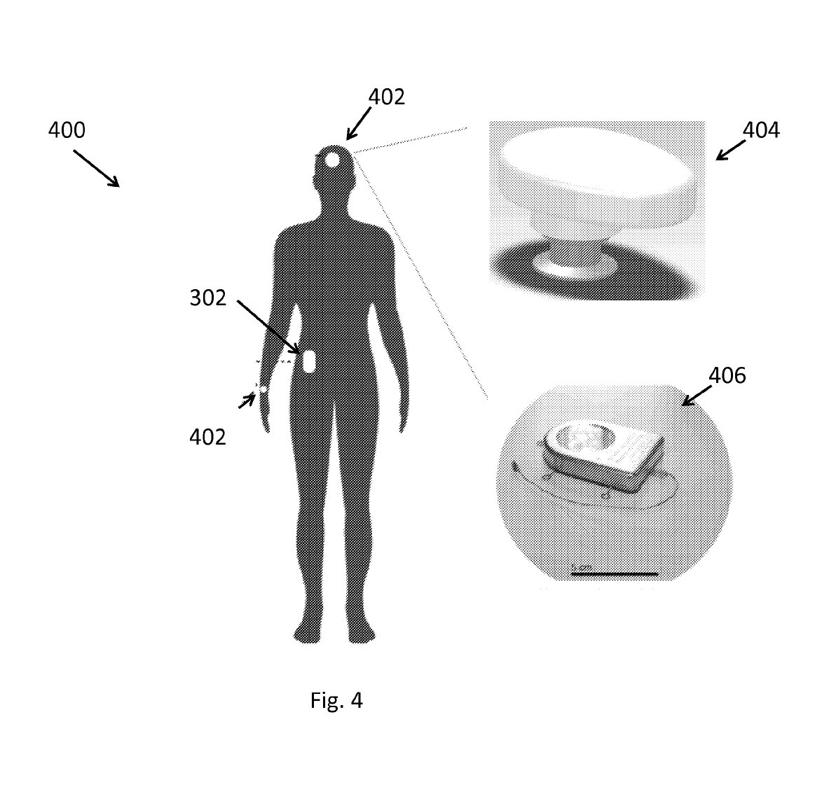

FIG. 4 provides additional details for the components of an exemplary WBMS 400, according aspects of the invention. The WBMS can include a wireless multichannel, broadband neural recording microsystem 402, which can include either an external head-mounted wireless module 402 or an implantable wireless module 404. The wireless multichannel, broadband neural recording microsystem 402 can digitize and broadcast the user's neural signals to the portable monitoring device 302. The portable monitoring device 302 can translate the received neural signals, for example, into control commands for a downstream effector, such as prosthetic hand 408. According to aspects of the disclosure, the WBMS can include more than one neural microsystems and the portable monitoring device can be configured to receive and process signals from all the neural microsystems in the WBMS.

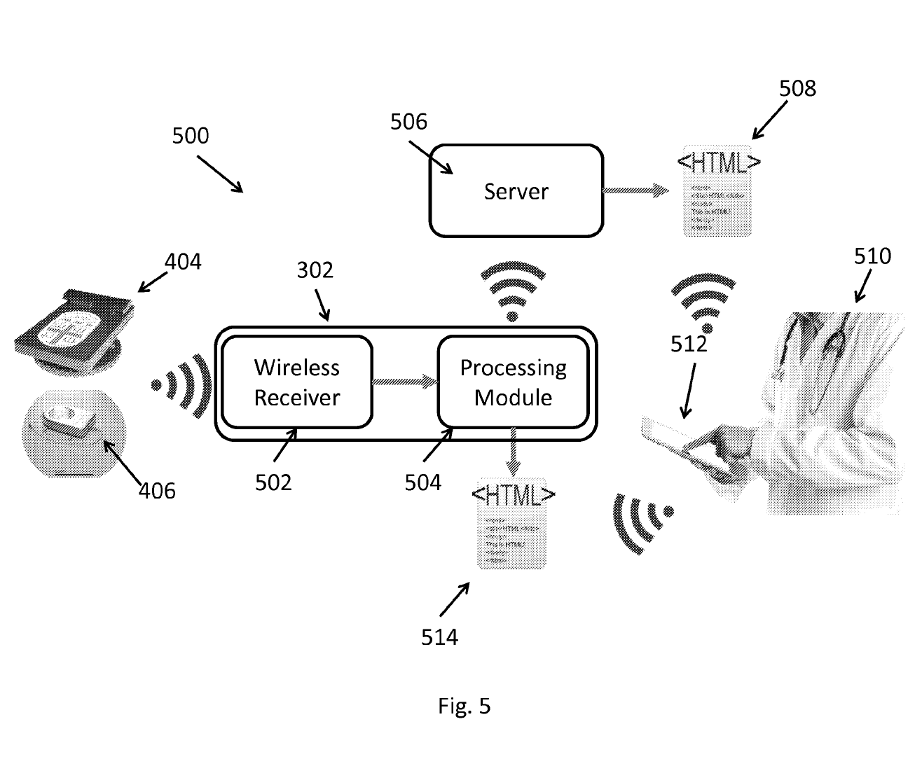

FIG. 5 provides an exemplary architecture 500 of the disclosed systems and methods for wireless applications, according to aspects of the disclosure. Neural data can be transmitted wirelessly from either an externally mounted neural recording device 404 or an implanted neural recording device 406 to a wireless receiver 502 of the portable monitoring device 302, which can receive the neural data and transfer them to a processor module 504 for processing. The wireless communication can be proprietary, for example, specific to the neural recording device, or an industry standard, such as IEEE 802.11. According to aspects of the disclosure, the wireless communication can be bidirectional if the neural recording device also contains neural stimulation capabilities or if the neural recording device accepts configuration commands. To decrease the memory requirements of the portable monitoring device 302, the portable monitoring device 302 can wirelessly transmit for example, via IEEE 802.11, raw or processed data to a server 506 for storage. The server 506 can host an HTML webpage 508 that can, for example, provide remote data visualization to a clinician 510. For example, a clinician can access and review the data from any internet-enabled device 512, such as a computer, tablet, or smartphone.

According to aspects of the disclosure, the portable monitoring device 302 can be reconfigurable. Configuration data for the portable monitoring device 302 can be sent through the server 506 as well by a clinician 510 when accessing the server 506 remotely. The portable monitoring device 302 can also host an HTML webpage 514 for the purpose of local access for system configuration. To ensure data security, all wireless communication can be encrypted. Additional security measures such as the use of biometrics or security keys, for example, radio frequency identification keys, during the configuration of the portable monitoring device 302 can also be used.

According to aspects of the disclosure, the disclosed systems and methods can accept wired and/or wireless digital or analog data from a variety of medical sensors and/or environmental sensors, and can interface with different systems using modular interfaces. For example, the medical sensor can be a heart rate sensor, a body temperature sensor, an oxygen blood sensor, a patient position sensor, an airflow sensor, an electrocardiogram sensor, a galvanic skin response sensor, a blood chemical sensor, an intracranial pressure sensor, a glucose sensor, and a respiratory effort sensor. For example, the environmental sensor can be a temperature sensor, an imaging sensor, a microphone, and a location sensor.

The disclosed system implements a real-time high-speed data processing architecture, which can be configurable and scalable. For example, the system can be implemented using field programmable gate arrays (FPGAs), which can be reprogramed to execute algorithms in hardware. Modern FPGAs are well-suited for portable devices, because they can consume low power, and therefore can be suitable for portable devices. In addition, FPGAs can be attached to data buses for expansion. Therefore, they can provide scalability and customizable computational capabilities. Specifically, the data processing architecture capabilities of the system can be expandable through the attachment of “daughter-cards” that can connect additional FPGAs to the system.

According to aspects of the disclosure, the whole-body monitoring system can be implemented in portable/wearable (“pocketable”) devices. The disclosed devices have a small packaging footprint and can be battery-powered. The disclosed devices can monitor their power usage and battery life, can monitor for failures, and can provide time estimates for battery recharging or replacement. Additionally, the portable devices can accept two separate batteries thereby allowing one battery to be replaced without the system powering off.

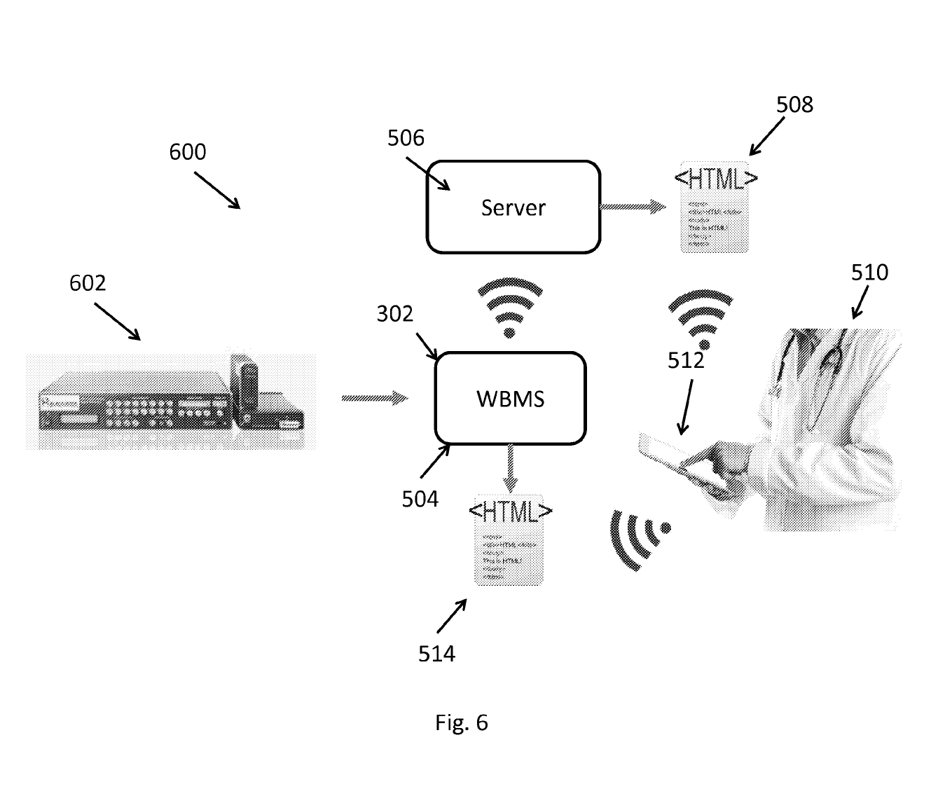

As illustrated in FIG. 5, neural data can be transmitted wirelessly to the WBMS 302. According to alternative aspects of the disclosure, neural data can also be transferred to the WBMS 302, through wired connections, as illustrated in FIG. 6. For example, neural data can be sensed by neural data sensors that are coupled to neural recording and analyzing systems 602, such as Blackrock Microsystems system. These systems 602 can be coupled to WBMS 302, which will process the neural data as described in the architecture of FIG. 5.

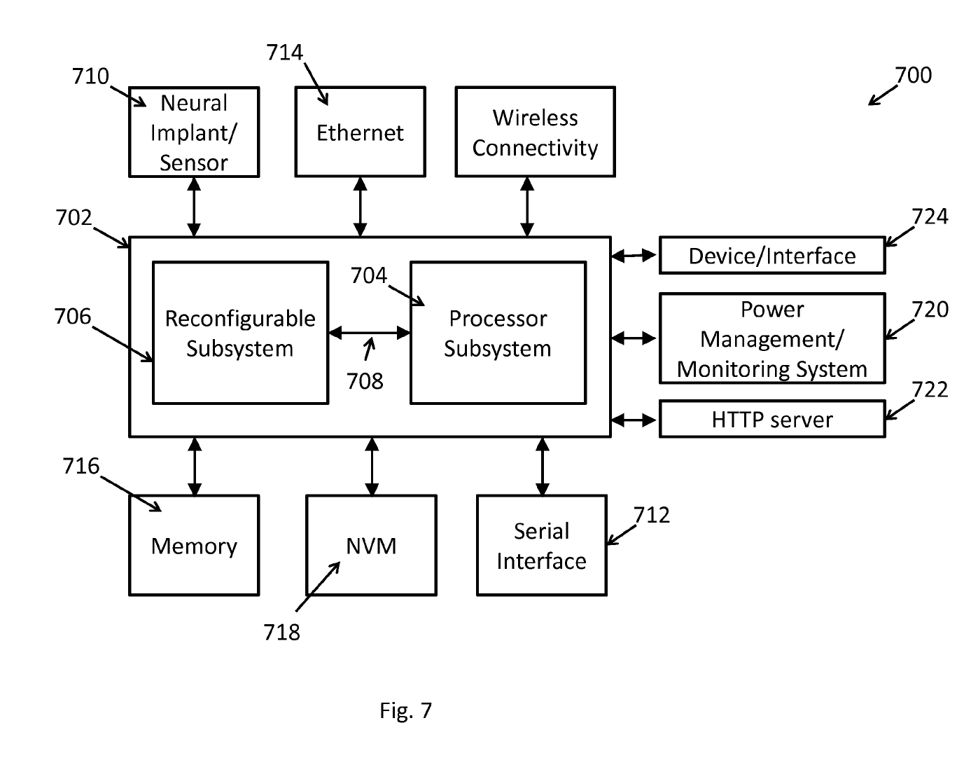

FIG. 7 shows an exemplary architecture 700 of a portable monitoring device, according to aspects of the disclosure. The portable monitoring device can include a system-on-a-chip (SoC) 702 that can combine a processor subsystem 704, such as an ARM processor, and a re-configurable subsystem 706, such as a Field-Programmable Gate Array (“FPGA”). These two subsystems can communicate within the SoC via high-bandwidth bus lines 708. The SoC 702 can include a receiver configured to receive data from a neural implant and/or other body sensor 710. According to aspects of the present disclosure, the portable monitoring device can also include an application-specific integrated circuit (ASIC) that can process the received data according a particular algorithm.

According to aspects of the disclosure, the processor subsystem 704 can be a programmable processor or a general-purpose processor. According to aspects of the disclosure, the processor subsystem 704 and the re-configurable subsystem 706 can be on the same integrated circuit or on separate integrated circuits.

According to other aspects of the disclosure, the SoC 702 can also include a transmitter configured to transmit data to the neural implant and/or other body sensor 710. The SoC 702 can process the received data and can communicate any processed data or results to other systems, for example, through a serial interface 712, such as USB or USB on-the go, or through Ethernet link 714, for example one or two gigabit Ethernet ports. The SoC 702 can also communicate any processed data or results wirelessly, for example, using IEEE 802.11 or Bluetooth.

The portable monitoring device can include memory 716, for example, a DDR3 SDRAM or a low power DDR3L-RS. The SoC 702 can communicate with the memory 716, which can host a real-time operating system, such as QNX, that can run on the processor subsystem 704 and can also operate as a data buffer. The portable monitoring device can also include non-volatile memory (NVM) 718, for example, NAND flash memory. Memory 716 can be used for running applications that can require high-speed memory transfers, while the NVM can be used for power-aware applications that can run using low-speed memory transfers.

The whole-body monitoring system can have a power management/monitoring system 702 that can be either integrated within the SoC 702 or can be implemented using discreet integrated circuits that can control the power to the SoC 702. The SoC 702 can also communicate with an HTTP/HTTPS server 722, which can be an interface for the user or their clinician to interact with the portable monitoring device. The SoC 702 can also be connected to various devices and interfaces 724, such as prosthetic arm, an assistive robot, a PC cursor or other controllable target devices. For example, the SoC 702 can send commands for controlling the devices/interfaces 724 according to signals received from the Neural Implant/Sensor 710. The SoC 702 can also be configured to interface with removable and/or stackable daughter cards that can add modular functionality to the whole-body monitoring system.

According to aspects of the disclosure, the processor subsystem 704 can receive peripheral device signals from peripheral devices, can send the received peripheral device signals to the re-configurable subsystem 706 for processing, receive, from the re-configurable subsystem 706, the processed peripheral device signals, and prepare the processed peripheral device signals for transmission to the at least one peripheral device.

The disclosed systems can act as data handling hub capable of broadcasting or storing raw or processed data. For example, it can broadcast data over the network, e.g., LANs or the Internet, or can store the data locally, e.g., in hard drives or USB drives.

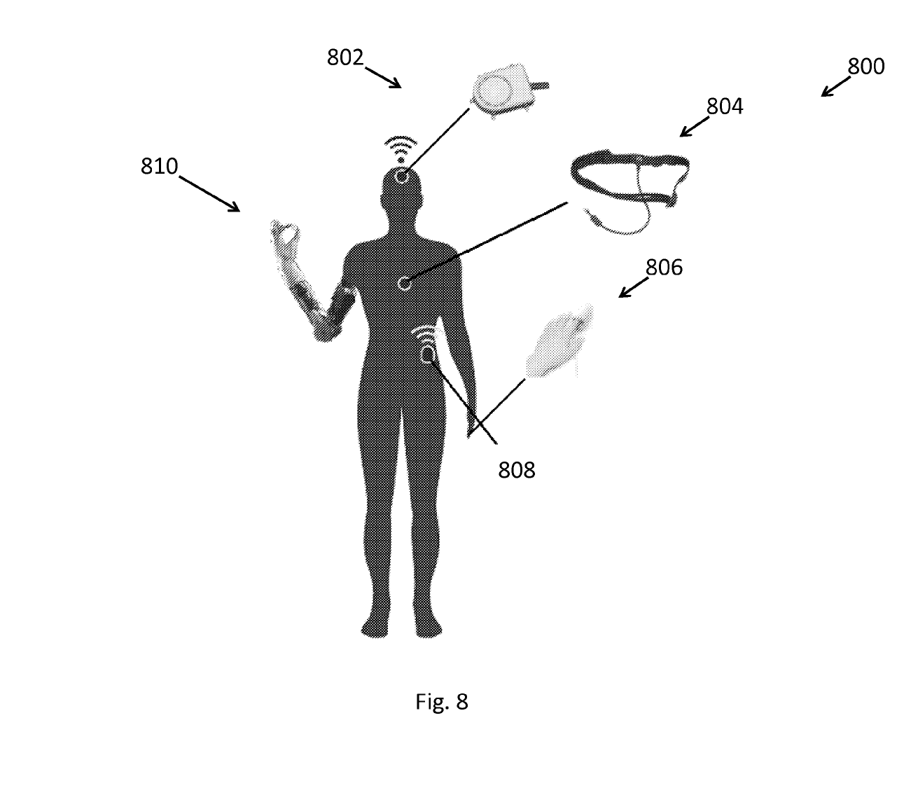

FIG. 8 shows exemplary components of a whole-body monitoring system according to aspects of the present disclosure. Specifically, FIG. 8 shows a wireless broadband neural sensor for monitoring broadband neural signals 802, a respiratory effort sensor 804, a heart rate sensor 806, a portable monitoring device 808, and a controllable target device 810. The disclosed systems and methods can support bidirectional communication links between the portable device and different sensors. The portable monitoring device 808 can receive signals from the different sensors, such as neural sensor 802, can process them, can map the processed signals into corresponding user-behaviors and user-intentions, and can generate actionable control commands, for example, for controllable target devices 810, e.g., mechanical prosthetic arm, wheelchair, functional electrical stimulation orthosis, computer/mobile device, deep brain stimulator (electrical and optogenetic stimulation), or devices for helping people with spinal cord injuries. The portable monitoring device 808 can also receive feedback data from the target devices 810. In addition, the portable monitoring device 808 can generate commands that can stimulate various body parts, e.g., muscles through electrical stimulation electrodes (internal or external) or brain, through electrical or optogenetic (light-based) stimulation. These commands can be transmitted either by wired or wireless connections.

A person of ordinary skill would understand that the portable monitoring device can generate commands and can interface with different target devices, for example, pacemakers, neural/muscle stimulators, amputee prosthetic devices, a functional electrical stimulator, a wheelchair, a computer, a smartphone, a tablet, a watch, a treadmill, a door, a car, a cochlear implant devices, and visual prosthetic devices, such as a retinal implant.

In addition, the portable monitoring device can transmit feedback or commands to the sensors, for example, to disable, wake up, or configure the operation mode of the sensors. For example, configuration options for the implant of the neural sensor can include, recording, stimulation, impedance, spectroscopy, low-power stand by. The disclosed portable monitoring device can provide an interface to the person to control the sensors, for example, through a touchscreen of a smartphone.

As explained above, the disclosed systems and methods are implemented on portable devices that have computation capacity to process in real-time the received signals and translate them into actionable control commands. Alternatively, the processing of the signals generated by the various sensors can be off-loaded to a server for processing. For example, the sensor data can be wirelessly transmitted to the server and when the data is processed at the server, it can be transmitted to the portable device for generating the control instructions.

The portable monitoring device can transmit to a remote location or store locally the unprocessed sensor data and/or the processed data for remote monitoring by a clinician or technician. For example, the system can allow a clinician to remotely monitor system user status, for example, heart rate, body temperature, respiratory rate, blood chemical concentrations, medicine dosages and delivery times, current medical device configurations. The data communications can be encrypted to ensure system user data privacy and security. The unprocessed and/or processed data can also be used for training the system to map the received unprocessed data to actionable commands for the target devices. The training of the model can utilize machine learning methods which generate a machine learning computational model given a set of training data.

As discussed above, the disclosed portable monitoring device can be implemented by a reconfigurable data processing architecture. The system can be reconfigured in real-time. The system can enable adjusting the data processing algorithms for a particular application or enable the implementation of an algorithm for new and different applications. For example, researchers and/or developers can configure the portable monitoring device at the system-level and implement custom algorithms for different applications.

According to aspects of the disclosure, the disclosed portable monitoring device can have a display to provide a visual interface to a user. If the portable monitoring device does not have a display, a secure HTTPS server communicating with the portable monitoring device can provide an interface to monitor or control the device, for example, through a website. For example, the website can allow clinicians and/or system users to configure the current algorithm, for example, change parameters, update the algorithm, for example, add, remove, or update the entire algorithm, view or download system user data, and monitor device power levels. All communication between the portable device and the server can be encrypted to prevent unauthorized persons from intercepting data or interacting with the device.

### Applications

The disclosed portable monitoring device can be used in a variety of applications and can be trained to generate different actionable commands in response to different identified patterned behaviors or applications. The portable monitoring device can be used, as discussed above, to help people suffering from particular diseases or injuries. However, healthy individuals can also use the system for general health monitoring purposes. The following discussion of the different applications is only exemplary of the capabilities of the disclosed system. A person of ordinary skill would understand that the portable monitoring device can be used for additional purposes and other application spaces.

Real-Time Epilepsy Detection and Suppression

The mobile and real-time aspects of the portable monitoring device can allow a person suffering from seizures to receive warnings for potential imminent seizures during the day and while performing normal everyday activities. For example, neural activity that is indicative of upcoming seizures can be decoded by the portable monitoring device and mapped to a particular pattern or condition, e.g., imminent seizure. In this case, for example, an appropriate control actionable command can be to issue a warning signal to the system user. Alternatively, the portable monitoring device can control a medical device connected to the system user to administer a drug dosage to the system user to prevent the seizure or ease its effects on the system user. The system can dispense an appropriate amount of medication automatically, either directly into a person or by communicating the appropriate dosage to an external medicine dispensing device. In addition, the system can also contact a medical emergency team or a doctor.

Such actionable commands can be particularly useful when the person is performing a task that could become catastrophic or cause serious effect, if a seizure were to occur. For example, if the system user received a warning about an imminent seizure while driving a car, the system user could pull off the road before the seizure started. The system could incorporate real-time weather reports to provide other types of recommendations, e.g. find shelter as soon as possible, or location information to identify the nearest hospital, where the system user could drive to.

As described above, the portable monitoring device can also provide neural stimulation to the brain, for example, by electrical, optical, and/or pharmacological means. In some cases this would allow the portable monitoring device to activate such stimulation to suppress seizures when possible.

### Closed-Loop Movement Disorders Control

According to aspects of the disclosure, the portable monitoring device can provide neural and/or muscular stimulation to control and/or mediate movement disorders, such as seizure suppressions. The portable monitoring device can provide information to a system user about past, present, and future states of their disorder, for example, to provide warnings of an impending seizure. The portable monitoring device can also allow remote monitoring of a system user's condition by clinician, and recommend scheduling a session with clinician depending on system user's current health status.

People with spinal cord injuries can benefit from the disclosed portable monitoring device. When an individual suffers a spinal cord injury, locomotion can be affected or, for a total transection, rendered entirely impossible. So long as the viability of the portion of the spinal cord below the transection has not been compromised, the portable monitoring device can restore control of the lower limbs. This can happen, for example, by circumventing the injured portion of the spine altogether, linking motor cortex activity directly to a spinal stimulator, for example, an implantable pulse generator (IPG) on the lower spine, where locomotor control is already hard-wired into the spine.

Raw neural data received by the portable monitoring device can be mapped and translated into control commands for a spinal stimulator. The portable monitoring device can implement algorithms that can estimate the phase of walking and can communicate, for example, via Wi-Fi or Bluetooth, appropriate stimulation parameters to the spinal simulator. This can cause the spinal cord stimulator to correct a disabled gait allowing for unimpeded walking.

The decoding algorithm can utilize machine learning techniques that can tune the algorithm's parameters based on training data. This training data can consist of the raw neural data, which constitute the machine learning features, and the corresponding phase in a subject's gait cycle, which constitutes the machine learning targets, measured using electromyography (EMG) sensors that can be placed on the subject. Once the parameters for the algorithm have been generated from training data, they can be dynamically loaded to the reconfigurable subsystem of the portable processing system, which can configure the algorithm into hardware in real-time.

People suffering from movement disorders, for example, Parkinson's disease or as a result of a stroke can receive improved treatment using a portable monitoring device. For example, the portable monitoring device can infer the level of undesired movement by recording and decoding, in real time, the brain's errant dynamics in the motor cortex. By monitoring the system user's neural activity, the portable monitoring device can provide targeted brain stimulation, for example, by electrical means that could inhibit undesired movement, for example, as shaking or trembling. This targeted brain stimulation can be adjusted in real-time. For example, the system user can receive a proper amount of neural stimulation at any given time to keep their side-effects suppressed. Using feedback control, the system can adjust the amount of neural stimulation and therefore can self-correct. The utilization of additional sensors, for example, inertial measurements sensors and other biomarkers, could further improve the portable monitoring device's ability to provide the optimal treatment, tailored to individual people.

In addition to Parkinson's disease, a person of ordinary skill would understand that the portable monitoring device can detect other moving disorders, such as, essential tremor, epileptic seizures, multiple sclerosis, and amyotrophic lateral sclerosis (ALS), and can provide insight to a system user and/or clinician about the disease state and its progression, or, in some instances, can treat the disease via neural stimulation, such as transcranial magnetic stimulation.

### Depression Treatment and Warning System

Under alternative embodiments, the portable monitoring device can monitor a person's depression. The portable monitoring device can provide both the system user and their clinician with a real-time description of the person's mental state as expressed through neural, physiological, and behavioral activity, for example, travel data collected via GPS or body movement via inertial measurement sensors.

When the portable monitoring device detects activity that indicates that the person is experiencing abnormal or severe depression, an appropriate actionable command could be to alert the appropriate authorities, if, for example, the person is located near a high-risk location, like a bridge. The portable monitoring device could locate the patent and understand the person's surroundings, for example, by utilizing its GPS capabilities.

The portable monitoring device can combine spatial information, for example, location of the person, as well as temporal information, for example, moving behavior, to make more accurate determinations of the users situation and intents. For example, a severely depressed person standing near a bridge for a long period of time could alert the authorities of a potential suicide risk with high probability. As discussed above, the portable monitoring device can incorporate real-time weather information or alerts to also provide other types of recommendations, for example identify a nearby shelter.

In addition to depression, a person of ordinary skill would understand that the portable monitoring device can detect other mood disorders, such as, schizophrenia, obsessive-compulsive disorder, and Tourette syndrome.

### Anxiety Monitoring System

A person's anxiety could be monitored constantly via neural recordings using the portable monitoring device providing both the system user and their clinician with a real-time description of the person's current anxiety level. Different situations can cause anxiety to a person, for example, being lost or in a new and unfamiliar location. The portable monitoring device can detect these situations and generate appropriate actionable commands. For example, when the portable monitoring device detects abnormal or high anxiety levels and also detects that the user is located in a location that is not among their usual locations, e.g., home or work, the portable monitoring device can alert a family member or display a link to a map with directions for the person how to return to their home. The portable monitoring device can contain a list of frequented locations to cross-reference person's current location to determine whether the person is lost or in an unfamiliar location.

A person of ordinary skill would understand that the system can be modified and trained to generate other commands that better fit the person's idiosyncrasy or habits. For example, the system can call a taxi service or dial an emergency number, for example, of a family member that the system user can talk to.

### Prosthetic or Auxiliary Devices Control

According to aspects of the disclosure, the portable monitoring device can provide a system user with the ability to control one or more prosthetic devices, a computer or smartphone, a wheelchair, various household appliances, a car, a functional electrical stimulator (artificial spinal cord), or an exoskeleton orthosis device. The portable monitoring device can send sensory information to the system user, for example, via physical transducers such as vibration mechanisms, or neural stimulation, such as a bi-directional brain-machine interface.

### Physical Therapy Monitoring

According to aspects of the disclosure, the portable monitoring device can provide a system user with live updates on a physical therapy progress. For example, the portable monitoring device can allow clinicians to monitor a system user's progress remotely. The portable monitoring device can inform a system user and/or clinician, for example, for medical issues and can also schedule an emergency medical session. The portable monitoring device can also provide means, such as an emergency button, to request for help if, for example, an injury occurs during therapy.

### Wireless Body Area Networks

According to aspects of the disclosure, the portable monitoring device can provide a system user with daily updates on specific aspects of their personal health, such as body temperature, heart rate, blood pressure, breathing rate, pulse, and blood chemical composition. In addition, the portable monitoring device can allow clinician to monitor system user's status remotely, can inform a system user to schedule session with a clinician, if any health indicator falls below a certain level, and can administer drugs either automatically or manually depending on current health state.

### Mood Disorders

According to aspects of the disclosure, the portable monitoring device can provide a system user with a breakdown of current mental state and can offer medication reminders and real-time medication adjustments. The portable monitoring device can also suggest scheduling an appointment with a clinician when necessary. In addition, the portable monitoring device can treat mood disorders with neural and/or muscular stimulation, for example, electrical, optical, and/or acoustic stimulation.

### Implant Management System

According to aspects of the disclosure, the portable monitoring device can provide intercommunication and data integration and processing capabilities to a system user with multiple medical implants and/or devices, such as an implanted EMG system and/or a neural system. The portable monitoring device can allow the implants/devices to operate more effectively and efficiently. For example, the portable monitoring device can synchronize pulmonary and cardiovascular implants to ensure optimal transfer of nutrients from the pulmonary system to the cardiovascular system. As another example, the portable monitoring device can increase the respiratory rate of an artificial lung implant to cause an increase in the volume of blood moved by an artificial heart. The portable monitoring device can manage various implant devices, such as a pulmonary implant, a cardiovascular implant, a drug delivery implant, a neural stimulator implant, a pacemaker, an intra-uterine device, a penile implant, an orthopedic implant, a defibrillator, a neural prosthesis, an insulin pump, an intrathecal pump, a visual prosthesis, a spinal prosthesis, an intracranial implant, and a cochlear implant

According to aspects of the disclosure, the portable monitoring device can generate and send stimulating signals back to the body, for example, to different muscles, and the brain of the system user, for example through deep brain stimulation via an optical stimulator or a chemical stimulator.

What is claimed is:

1. A portable monitoring device comprising:
a receiver configured to:
receive wireless signals representing real-time neural activity from a neural sensor configured to sample neural signals from a person; and
process the wireless signals into corresponding digital signals;
a first processor configured to:
receive the digital signals from the receiver;
process the digital signals to generate processed digital signals; and
generate a mapping of the neural activity into at least one of a person's behavior, a person's mood, a person's health condition, a person's memory, and a person's intentions; and
a second processor coupled to the first processor configured to:
receive the digital signals and the processed digital signals data from the first processor; and
prepare at least one of the digital signals and the processed digital signals data for transmission to at least one peripheral device coupled to the portable monitoring device.

2. The portable monitoring device of claim 1, wherein at least one of the first processor and the second processor is a programmable processor.

3. The portable monitoring device of claim 1, wherein the first processor and the second processor are on a same integrated circuit.

4. The portable monitoring device of claim 1, wherein the first processor is a field programmable gate array and the second processor is a general purpose processor.

5. The portable monitoring device of claim 1, wherein the second processor is further configured to:
receive peripheral device signals from the at least one peripheral device;
send the received peripheral device signals to the first processor for processing;
receive, from the first processor, the processed peripheral device signals; and
prepare the processed peripheral device signals for transmission to the at least one peripheral device.

6. The portable monitoring device of claim 1, further comprising a user interface configured to display a representation of the at least one of a person's behavior, a person's mood, a person's health condition, a person's memory, and a person's intentions.

7. The portable monitoring device of claim 1, further comprising a memory unit and wherein the first processor is configured to:
store the digital signals into the memory unit; and
transfer the stored digital signals from the memory unit to the second processor.

8. The portable monitoring device of claim 1, further comprising a memory unit and wherein the second processor is configured to:
store the digital signals into the memory unit; and
transfer the stored digital signals from the memory unit to the first processor.

9. The portable monitoring device of claim 1, wherein the portable monitoring device is coupled to a server and wherein at least one of the first and second processor is configured to transfer the digital signals to the server.

10. The portable monitoring device of claim 1, further comprising a wireless transmitter configured to transmit configuration data to the neural sensor.

11. The portable monitoring device of claim 1, wherein the receiver is further configured to:
receive second wireless signals from at least one of a medical sensor and an environmental sensor; and
process the second wireless signals into corresponding second digital signals.

12. The portable monitoring device of claim 11, wherein at least one of the first and second processor is further configured to:
process the second digital signals; and
generate an interpretation of at least one of a person's behavior, a person's mood, a person's health condition, a person's memory, and a person's intentions, based on the digital signals and second digital signals.

13. The portable monitoring device of claim 12, wherein the medical sensor is at least one of a heart rate sensor, a body temperature sensor, an oxygen blood sensor, a patient position sensor, an airflow sensor, an electrocardiogram sensor, a galvanic skin response sensor, a blood chemical sensor, an intracranial pressure sensor, a glucose sensor, and a respiratory effort sensor and wherein the environmental sensor is at least one of a temperature sensor, an imaging sensor, a microphone, and a location sensor.

14. The portable monitoring device of claim 1, wherein at least one of the first processor and the second processor is further configured to generate at least one command in response to the mapping of the neural activity for controlling a target device coupled to the portable monitoring device.

15. The portable monitoring device of claim 14, wherein the target device comprises at least one of a neural stimulator, a muscle stimulator, a pacemaker, an amputee prosthetic device, a cochlear implant device, a functional electrical stimulator, a wheelchair, a computer, a smartphone, a tablet, a watch, a treadmill, a door, a car, and a visual prosthetic device.

16. The portable monitoring device of claim 1, wherein the receiver is further configured to:
receive wireless second signals from an implant device; and
process the second wireless signals into corresponding second digital signals.

17. The portable monitoring device of claim 16, wherein at least one of the first processor and the second processor is further configured to:
process the second digital signals; and
generate configuration data for the implant device based on at least one of the processed first digital signals and the processed second digital signals; and
wherein the portable monitoring device further comprising a wireless transmitter configured to transmit the configuration data to the implant device.

18. The portable monitoring device of claim 17, wherein the implant device is at least one of a pulmonary implant, a cardiovascular implant, a drug delivery implant, a neural stimulator implant, a pacemaker, an intra-uterine device, a penile implant, an orthopedic implant, a defibrillator, a neural prosthesis, an insulin pump, an intrathecal pump, a visual prosthesis, a spinal prosthesis, an intracranial implant, and a cochlear implant.

19. The portable monitoring device of claim 1, wherein the portable monitoring device can be re-configured over a network interface.

20. A method for whole body monitoring using a portable monitoring device comprising:
receiving, by a receiver of the portable monitoring device, wireless signals representing real-time neural activity from a neural sensor configured to sample neural signals from a person;
processing, by the receiver, the wireless signals into corresponding digital signals;
receiving, by a first processor of the portable monitoring device, the digital signals from the receiver;
processing, by the first processor, the digital signals to generate processed digital signals;
generating, by the first processor, a mapping of the neural activity into at least one of a person's behavior, a person's mood, a person's health condition, a person's memory, and a person's intentions;
receiving, by a second processor of the portable monitoring device coupled to the first processor, the digital signals and the processed digital signals data from the first processor; and
preparing, by the second processor, at least one of the digital signals and the processed digital signals data for transmission to at least one peripheral device coupled to the portable monitoring device.

21. The method of claim 20, further comprising, generating, by at least one of the first processor and the second processor, at least one command in response to the mapping of the neural activity for controlling a target device coupled to the portable monitoring device.

22. The method of claim 21, wherein the target device comprises at least one of a neural stimulator, a muscle stimulator, a pacemaker, an amputee prosthetic device, a cochlear implant device, a functional electrical stimulator, a wheelchair, a computer, a smartphone, a tablet, a watch, a treadmill, a door, a car, and a visual prosthetic device.

23. The method of claim 20, further comprising:
receiving peripheral device signals from the at least one peripheral device;
sending the received peripheral device signals to the first processor for processing;
receiving, from the first processor, the processed peripheral device signals; and
preparing the processed peripheral device signals for transmission to the at least one peripheral device.

24. The method of claim 20, further comprising:
receiving second wireless signals from at least one of a medical sensor and an environmental sensor; and
processing the second wireless signals into corresponding second digital signals.

25. The method of claim 24, further comprising
processing the second digital signals; and
generating an interpretation of at least one of a person's behavior, a person's mood, a person's health condition, a person's memory, and a person's intentions, based on the digital signals and second digital signals.

26. The method of claim 20, further comprising:
receiving wireless second signals from an implant device; and
processing the second wireless signals into corresponding second digital signals.

27. The method of claim 26, further comprising:
processing the second digital signals; and
generating configuration data for the implant device based on at least one of the processed first digital signals and the processed second digital signals; and
wherein the portable monitoring device comprises a wireless transmitter configured to transmit the configuration data to the implant device.

28. The method of claim 27, wherein the implant device is at least one of a pulmonary implant, a cardiovascular implant, a drug delivery implant, a neural stimulator implant, a pacemaker, an intra-uterine device, a penile implant, an orthopedic implant, a defibrillator, a neural prosthesis, an insulin pump, an intrathecal pump, a visual prosthesis, and a cochlear implant.
